(19) United States
(12) Patent Application Publication
(10) Pub. No.: US 2011/0253984 A1
(43) Pub. Date: Oct. 20, 2011

(54) ELECTRONIC GRADE SILK SOLUTION, OTFT AND MIM CAPACITOR WITH SILK PROTEIN AS INSULATING MATERIAL AND METHODS FOR MANUFACTURING THE SAME

(76) Inventors: Jenn-Chang HWANG, Hsinchu (TW); Chung Hwa Wang, Hsinchu (TW); Chao Ying Hsieh, Hsinchu (TW)

(21) Appl. No.: 12/841,678

(22) Filed: Jul. 22, 2010

Related U.S. Application Data
(63) Continuation-in-part of application No. 12/761,008, filed on Apr. 15, 2010.

(51) Int. Cl. H01L 51/30; C07K 14/435; B05D 5/12; H01L 51/42; H01G 4/14

(52) U.S. Cl. 257/40; 438/99; 361/311; 427/77; 530/353; 257/E51.006; 257/E51.045

(57) ABSTRACT

An electronic grade silk solution, an organic thin film transistor (OTFT) and a metal-insulator-metal capacitor with silk protein as the insulating material manufactured by use of the silk solution, and methods for manufacturing the same are disclosed. The OTFT of the present invention comprises: a substrate; a gate disposed on the substrate; a gate insulating layer containing silk protein, which is disposed on the substrate and covers the gate; an organic semiconductor layer; and a source and a drain, wherein the organic semiconductor layer, the source and the drain are disposed over the gate insulating layer.

### CROSS REFERENCE TO RELATED APPLICATION

This application is a continuation-in-part application, and claims benefit of U.S. patent application Ser. No. 12/761,008, filed Apr. 15, 2010, entitled “OTFT AND MIM CAPACITOR USING SILK PROTEIN AS DIELECTRIC MATERIAL AND METHODS FOR MANUFACTURING THE SAME,” by Jenn-Chang Hwang, Chung Hwa Wang, and Chao Ying Hsieh, which status is pended, the disclosure of which is hereby incorporated herein in its entirety by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### BACKGROUND OF THE INVENTION

1. Field of the Invention

The present invention relates to an electronic grade silk solution, an organic thin film transistor (OTFT) and a metal-insulator-metal (MIM) capacitor with silk protein as an insulating material using the electronic grade silk solution, and methods for manufacturing the same and, more particularly, to an electronic grade silk solution, an OTFT and a MIM capacitor using silk protein as an insulating material, and methods for manufacturing the same through a solution process.

2. Description of Related Art

Thin film transistors (TFTs) are fundamental components in contemporary electronics, such as sensors, radio frequency identification (RFID) tags, and electronic display devices. In recent years, in order to reduce the production cost and increase the product application, organic thin film transistors (OTFTs) have been rapidly developed which have the advantages of low-cost and flexibility, and can be produced in large-area.

OTFTs can be fabricated using either bottom gate or top gate architectures. In the bottom gate architecture, OTFTs can be divided into top contact OTFTs and bottom contact OTFTs. As shown in FIG. 1A, the top contact OTFT comprises: a substrate 10; a gate electrode 11 disposed on the substrate 10; a gate insulating layer 12 disposed on the substrate 11 and covering the gate electrode 11; an organic semiconductor layer 13 covering the entire surface of the organic semiconductor layer 12; and a source electrode 14 and a drain electrode 15 disposed on the organic semiconductor layer 13 respectively.

In addition, as shown in FIG. 1B, the bottom contact OTFT comprises: a substrate 10; a gate electrode 11 disposed on the substrate 10; a gate insulating layer 12 disposed on the substrate 10 and covering the gate electrode 11; a source electrode 14 and a drain electrode 15 disposed on the gate insulating layer 12 respectively; and an organic semiconductor layer 13 covering the gate insulating layer 12, the source electrode 14, and the drain electrode 15.

In the conventional method for forming the gate insulating layer, the dielectric material is sputtered on the substrate and the gate electrode to form the gate insulating layer. However, the instrument for the sputtering process is very expensive and the process is complex. In addition, the most suitable material conventionally used in the organic semiconductor layer of the OTFT is pentacene. However, pentacene cannot match well with the conventional dielectric material, so the carrier mobility of pentacene is low. For example, when silicon nitride is used as a material of the gate insulating layer in the pentacene OTFT, the carrier mobility of the pentacene is lower than 0.5 cm²N-sec. Even though aluminum nitride is used as the material of the gate insulating layer in the pentacene OTFT, the carrier mobility of the pentacene cannot be higher than 2 cm²/V-sec. Hence, it is impossible to manufacture OTFTs with high performance by using the present techniques and materials.

Therefore, it is desirable to develop an OTFT and a method for manufacturing the same, in order to prepare OTFTs in a simple and cheap way, and increase the performance of the OTFTs.

In addition, the metal-insulator-metal (MIM) capacitors are widely applied on digital and radio frequency (RF) circuit designs. Currently, several dielectric materials with high dielectric constant are developed to increase the capacitor density of the MIM capacitors and decrease the leakage current thereof.

As shown in FIG. 2, a conventional MIM capacitor comprises: a substrate 20; a first electrode 21 disposed on the substrate 20; an insulating layer 22, disposed on the substrate 20 and covering the first electrode 21; and a second electrode 22 disposed on the insulating layer 22. Herein, the conventional dielectric material used in the insulating layer of the MIM capacitor can be TiN, TiO₂, SiO₂, and SiN. However, when the aforementioned dielectric material is used as the insulating layer of the MIM capacitor, the insulating layer is formed on the metal layer by use of the sputtering process or the vacuum deposition equipment, which may cause the production cost and the process complexity to be increased.

Therefore, it is desirable to develop a MIM capacitor and a method for manufacturing the same, in order to prepare MIM capacitors in a simple and cheap way and to apply them on various digital and RF circuits. In addition, when the conventional dielectric material is used, a sputtering process or a vacuum deposition process has to be performed to form the gate insulating layer of the OTFT and the insulating layer of the MIM capacitor. Hence, the complexity of the process and the production cost are greatly increased. Therefore, it also desirable to develop a dielectric material for electronic devices, which can be easily available, manufactured in a simple process, and applied to several electronic devices.

### SUMMARY OF THE INVENTION

The object of the present invention is to provide an OTFT and a method for manufacturing the same to prepare an OTFT with high performance.

Another object of the present invention is to provide a MIM capacitor and a method for manufacturing the same, in order to prepare a MIM capacitor through a simple and cheap process.

A further object of the present invention is to provide an electronic grade silk solution. When the electronic grade silk solution of the present invention is used, it is possible to obtain electronic devices with silk protein as a dielectric material.

To achieve the object, the OTFT of the present invention comprises: a substrate; a gate electrode disposed on the substrate; a gate insulating layer disposed on the substrate and covering the gate electrode, wherein the gate insulating layer comprises silk protein; an organic semiconductor layer; and a source electrode and a drain electrode, wherein the organic semiconductor layer, the source electrode, and the drain electrode are disposed over the gate insulating layer.

In addition, the present invention further provides a method for manufacturing the aforementioned OTFT, which comprises the following steps: (A) providing a substrate; (B) forming a gate electrode on the substrate; (C) coating the substrate having the gate electrode formed thereon with a silk solution to obtain a gate insulating layer on the substrate and the gate electrode; and (D) forming an organic semiconductor layer, a source electrode, and a drain electrode over the gate insulating layer.

Furthermore, in order to achieve the object, the MIM capacitor of the present invention comprises: a substrate; a first electrode disposed on the substrate; an insulating layer disposed on the substrate and covering the first electrode, wherein the insulating layer comprises silk protein; and a second electrode disposed on the insulating layer.

In addition, the present invention further provides a method for manufacturing the aforementioned MIM capacitor, which comprises the following steps: (A) providing a substrate; (B) forming a first electrode on the substrate; (C) coating the substrate having the first electrode formed thereon with a silk solution to obtain an insulating layer on the substrate and the first electrode; and (D) forming a second electrode on the insulating layer.

Furthermore, the present invention also provides an electronic grade silk solution, which comprises silk protein and water, wherein the pH of the electronic grade silk solution is 3˜6. In addition, the present invention also provides a method for manufacturing the aforementioned electronic grade silk solution, which comprises the following steps: (A) placing a silkworm cocoon into an alkali solution, and heating the alkali solution to obtain silk protein; (B) dissolving the silk protein in a phosphoric acid solution; and (C) removing the phosphate in the phosphoric acid solution dissolving with the silk protein to obtain a silk solution.

According to the OTFT and the MIM capacitor and the methods for manufacturing the same of the present invention, the substrate with a gate electrode or a first electrode formed thereon is coated with a silk solution to form a gate insulating layer or an insulating layer containing silk protein. Compared to the conventional method for forming the gate insulating layer or an insulating layer through a sputtering process or a vacuum deposition process, the method of the present invention can be performed in a solution process. Hence, the process of the present invention is low cost and simple, and can be used for preparing the OTFT and the MIM capacitor with large area. Also, the silk protein is low cost and easily available. In addition, the silk protein used in the OTFT of the present invention matches well with the material of the organic semiconductor layer, so the transistor characteristics of the OTFT can be greatly improved. Furthermore, according to the electronic grade silk solution and the method for manufacturing the same of the present invention, a silk solution suitable for various electronic devices can be obtained through a simple and cheap process. Also, the insulating layer or the dielectric layer of the electronic device can be obtained through a solution process by use of the electronic grade silk solution of the present invention. Hence, the production cost and the process complexity can be greatly decreased.

According to the electronic grade silk solution and method for manufacturing the same, and the structure the OTFT and the MIM capacitor of the present invention, the silk protein may be natural silk protein. Preferably, the silk protein is fibroin. In addition, according to the methods for manufacturing the OTFT and the MIM capacitor of the present invention, the silk solution may be an aqueous solution containing natural silk protein. Preferably, the silk solution is an aqueous solution containing fibroin.

According to the method for manufacturing an OTFT of the present invention, the step (C) for coating the silk solution may further comprise the following steps: (C1) providing a silk solution; (C2) coating the substrate having the gate electrode formed thereon with the silk solution; and (C3) drying the silk solution coated on the substrate and the gate electrode to obtain a gate insulating layer on the substrate and the gate electrode. Herein, the step (C2) can be performed through a spin coating process, a dip coating process, a roll coating process, or a printing process. Preferably, the step (C2) is performed through a dip coating process. Hence, the step (C2) is preferably performed by immersing the substrate having the gate electrode formed thereon into the silk solution to coat the substrate and the gate electrode with the silk solution.

In addition, according to the method for manufacturing a MIM capacitor of the present invention, the step (C) for coating the silk solution may further comprise the following steps: (C1) providing a silk solution; (C2) coating the substrate having the first electrode formed thereon with the silk solution; and (C3) drying the silk solution coated on the substrate and the first electrode to obtain an insulating layer on the substrate and the first electrode. Herein, the step (C2) can be performed through a spin coating process, a dip coating process, a roll coating process, or a printing process. Preferably, the step (C2) is performed through a dip coating process. Hence, the step (C2) is preferably performed by immersing the substrate having the first electrode formed thereon into the silk solution to coat the substrate and the first electrode with the silk solution.

Hence, according to the methods for manufacturing the OTFT and the MIM capacitor of the present invention, the silk film, which is used as a gate insulating layer or an insulating layer, can be easily formed through a dip coating process and a drying process. Herein, the drying process can be any conventional drying method, such as an air-drying process or a baking process. When the step (C) for coating the silk solution is performed one time, the silk film with a single-layered structure is obtained. In addition, the step (C) can be repeated to form the silk film with a multi-layered structure, if it is needed.

In addition, according to the OTFT and the MIM capacitor and the methods for manufacturing the same of the present invention, the substrate may be a plastic substrate, a glass substrate, a quartz substrate, or a silicon substrate. Preferably, the substrate is a flexible substrate, i.e. a plastic substrate. When the plastic substrate is used, the device prepared in the present invention has flexibility. In addition, the material of each electrodes containing the gate electrode, the source electrode, the drain electrode, the first electrode and the second electrode may be independently selected from the group consisting of Cu, Cr, Co, Ni, Zn, Ag, Pt, Au, and Al.

According to the OTFT and the method for manufacturing the same of the present invention, the material of the organic semiconductor layer may comprise pentacene, and other suitable materials. Preferably, the material of the organic semiconductor layer is pentacene.

Furthermore, according to the method for manufacturing the OTFT of the present invention, the organic semiconductor layer covers the entire surface of the gate insulating layer, and the source electrode and the drain electrode are formed on the organic semiconductor layer to obtain a top contact organic thin film transistor, in the step (D).

In addition, according to the method for manufacturing the OTFT of the present invention, the source electrode and the drain electrode are formed on the gate insulating layer, and the organic semiconductor layer covers the source electrode, the drain electrode, and the gate insulating layer to obtain a bottom contact organic thin film transistor, in the step (D).

Furthermore, according to the electronic grade silk solution and method for manufacturing the same, the step (C) may be: dialyzing the phosphoric acid, which is dissolved with the silk protein, with water to remove phosphate to obtain a silk solution. Herein, the dialysis process can be performed by placing the phosphoric acid solution dissolving with the silk protein into a membrane. The molecular weight cutoff of the membrane can be 10000˜20000. Preferably, the molecular weight cutoff of the membrane is 12000˜16000. More preferably, the molecular weight cutoff of the membrane is 13000˜45000. In addition, the step (A) may further comprise the following steps (A1) to (A3): (A1) placing the silkworm cocoon into an alkali solution, and boiling the alkali solution to obtain silk protein; (A2) taking out the silk protein from the alkali solution, and washing the silk protein; and (A3) drying the silk protein after washing. Preferably, the alkali solution is a solution of sodium carbonate.

### BRIEF DESCRIPTION OF THE DRAWINGS

FIG. 1A is a perspective view of a conventional top contact OTFT;

FIG. 1B is a perspective view of a conventional bottom contact OTFT;

FIG. 2 is a perspective view of a conventional MIM capacitor;

FIGS. 3A to 3D are cross-sectional views illustrating the process for manufacturing a top contact OTFT in Embodiment 1 of the present invention;

FIG. 4 is a curve showing the transfer characteristic of the OTFT of Embodiment 1 of the present invention;

FIG. 5 is a curve showing the output characteristic of the OTFT of Embodiment 1 of the present invention;

FIGS. 6A to 6C are cross-sectional views illustrating the process for manufacturing a bottom contact OTFT in Embodiment 2 of the present invention;

FIGS. 7A to 7C are cross-sectional views illustrating the process for manufacturing a MIM capacitor in Embodiment 3 of the present invention;

FIG. 8 is a curve showing the capacitance-frequency characteristic of the MIM capacitor of Embodiment 3 of the present invention; and

FIG. 9 is a curve showing the electric field-leakage current characteristic of the MIM capacitor of Embodiment 3 of the present invention.

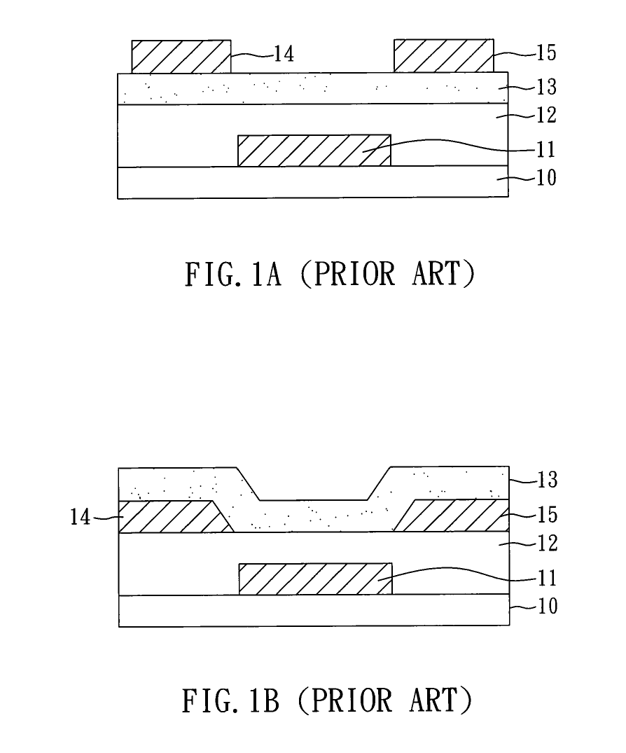

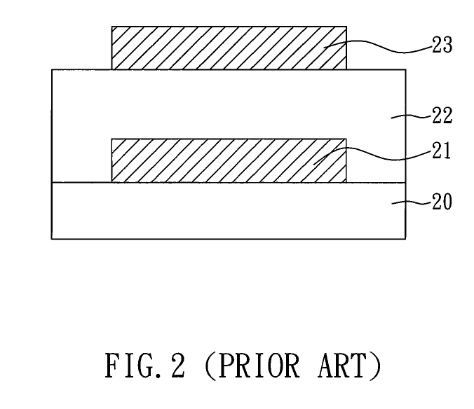

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Embodiment 1

### Top Contact OTFT

### [Preparation of a Silk Solution]

First, 10 wt % of an aqueous solution of Na₂CO₃ was provided and heated. When the solution was boiling, silkworm cocoon (natural silk) was added thereto, and the solution was kept boiling for 30 min to 1 hr to remove sericin. Then, the silk without sericin was washed by deionized water to remove the alkali salt adhered on the silk. After a drying process, refined silk, i.e. fibroin, was obtained.

Next, the refined silk was added into 85 wt % of phosphoric acid (H₃PO₄) solution (20 ml), and the resulted solution was stirred until the refined silk was dissolved. Then, the phosphoric acid solution containing the refined silk was put into a membrane (Spectra/Por 3 membrane, molecular weight cutoff=14000) and dialyzed with water. The dialysis process was performed for 3 days to remove the phosphate ions. The pH of the resulting silk solution can be adjusted by changing the volume of the water and the number of times of the dialysis process. Herein, the pH of the resulting silk solution is controlled between 3 to 6. After the dialysis process is completed, a filter paper is used to filter out impurities, and an aqueous solution of fibroin is obtained.

### [Preparation of a Top Contact OTFT]

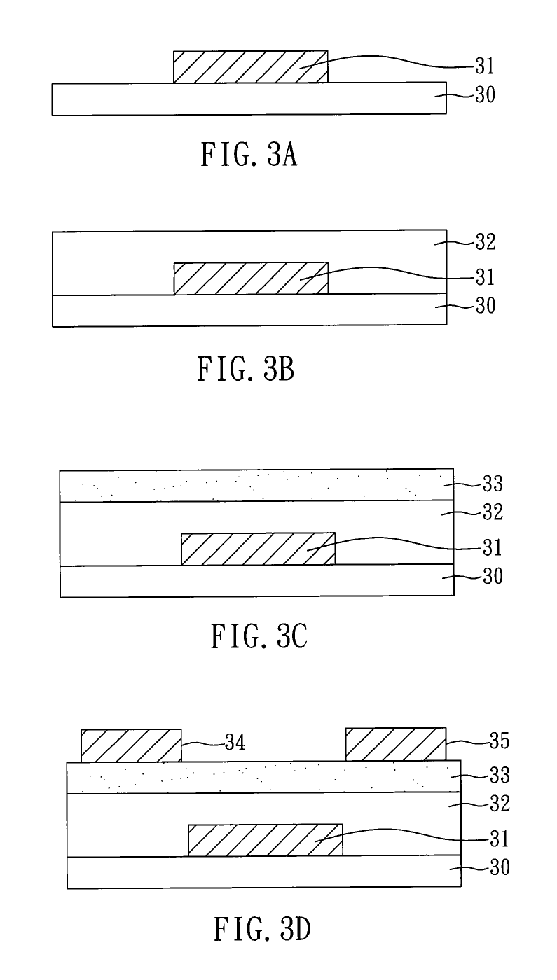

As shown in FIG. 3A, a substrate 30 was provided, and the substrate 30 was cleaned by deionized water through a sonication process. In the present embodiment, the substrate 30 was a plastic substrate made of PET.

Next, the substrate 30 was placed inside a vacuum chamber (not shown in the figure), and a metal was evaporated on the substrate 30 by using a mask (not shown in the figure) to form a patterned metal layer, which was used as a gate electrode 31, as shown in FIG. 3A. In the present embodiment, the metal used in the gate electrode 31 was Au, and the thickness of the gate electrode 31 was about 80 nm. In addition, the condition of the evaporation process for forming the gate electrode 31 is listed below.
Pressure: 5×10⁻⁶ torr
Evaporation rate: 1 Å/s

Then, the substrate 30 having the gate electrode 31 formed thereon was dipped into the silk solution for 15 mins to coat the substrate 30 having the gate electrode 31 with the silk solution. After the coating process, the substrate 30 coated with the silk solution was dried at 60° C. to form a silk film, and the silk film was used as a gate insulating layer 32, as shown in FIG. 3B. In the present embodiment, the gate insulating layer 32 formed by the silk film has a thickness of 400 nm. In addition, the coating process and the drying process can be performed several times to form a silk film with multi-layered structure.

As shown in FIG. 3C, through a heat evaporation process, pentacene was deposited on the gate insulating layer 32 at room temperature by use of a shadow metal mask to form an organic semiconductor layer 33. In the present embodiment, the thickness of the organic semiconductor layer 33 is about 70 nm. In addition, the condition of the heat evaporation process for forming the organic semiconductor layer 33 is listed below.
Pressure: 2×10⁻⁶ torr
Evaporation rate: 0.3 Å/s

Finally, the same evaporation process for forming the gate electrode was performed to form a patterned metal layer, which was used as a source electrode 34 and a drain electrode 35, on the organic semiconductor layer 33 by using another mask (not shown in the figure), as shown in FIG. 3D. In the present embodiment, the material of the source electrode 34 and the drain electrode 35 was Au, and the thickness of source electrode 34 and the drain electrode 35 was about 80 nm.

As shown in FIG. 3D, after the aforementioned process, a top contact OTFT of the present embodiment was obtained, which comprises: a substrate 30; a gate electrode 31 disposed on the substrate 30; a gate insulating layer 32 disposed on the substrate 30 and covering the gate electrode 31, wherein the gate insulating layer 32 comprises silk fibroin; an organic semiconductor layer 33 covering the entire surface of the gate insulating layer 32; and a source electrode 34 and a drain electrode 35, respectively disposed on the organic semiconductor layer 33.

### [Evaluation the Characteristics of the OTFT]

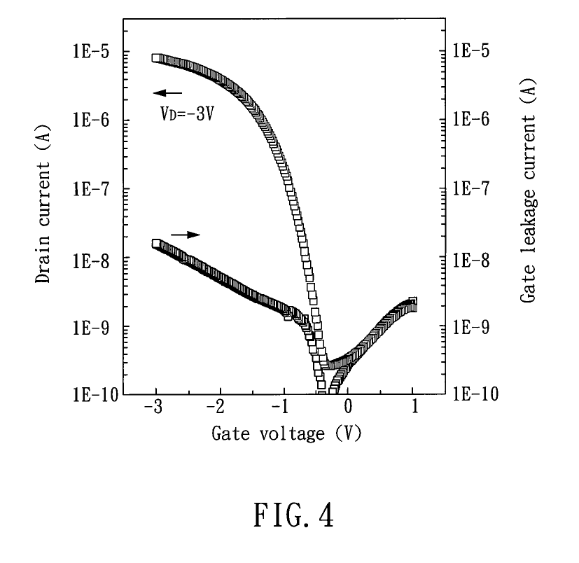

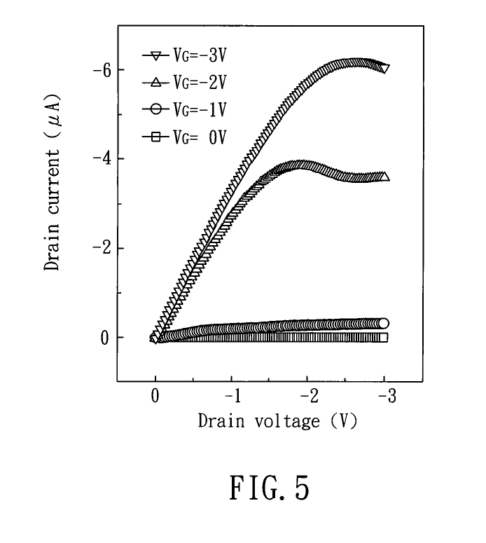

A current-voltage test was performed on the top contact OTFT of the present embodiment. The result of the transfer characteristic of the OTFT is shown in FIG. 4, and the result of the output characteristic under different gate voltage (V_{G}) is shown in FIG. 5. The current on-to-off ratio (I_{ON/OFF}), the subthreshold swing (S,S), the carrier mobility and the threshold voltage (V_{TH}) are listed in the following Table 1.

TABLE 1

| | Results | |
|---|---|---|
| Channel width | 600 | μm |
| Channel length | 75 | μm |
| Thickness of the gate insulating layer | 400 | nm |
| Thickness of the organic semiconductor layer | 70 | nm |
| I_{ON/OFF} | 4 × 10⁴ | |
| S.S | 172 | mV/decade |
| Carrier mobility | 40.1 | cm²/V-sec |
| V_{TH} | −0.76 | V |

According to the results shown in FIG. 4, FIG. 5 and Table 1, the carrier mobility of the gate insulating layer made of the silk protein is about 40 cm²/V-sec. Compared to the conventional pentacene OTFT with a gate insulating layer made from silicon nitride or aluminum nitride, the device performance of the pentacene OTFT of the present embodiment can be improved greatly, due to use of the silk protein as a dielectric material of the gate insulating layer.

### Embodiment 2

### Bottom Contact OTFT

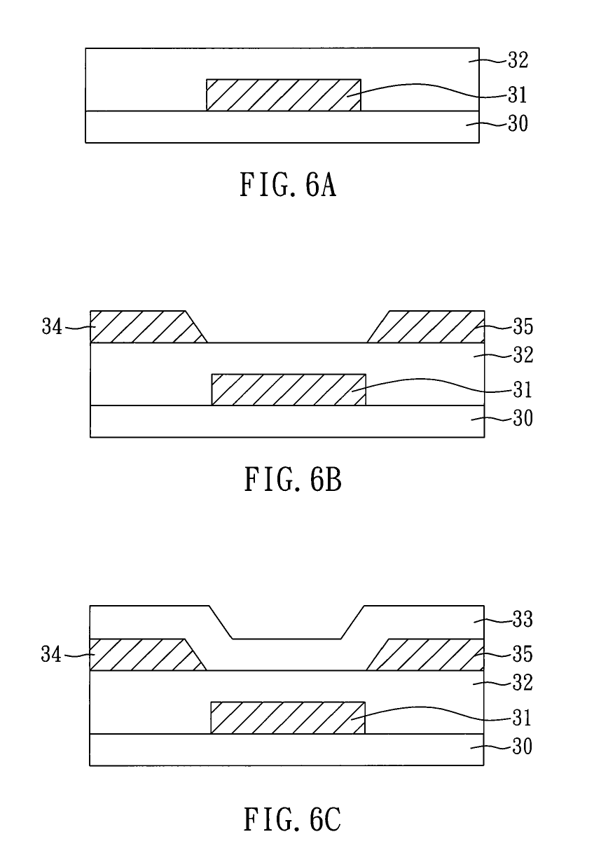

As shown in FIG. 6A, a substrate 30 was provided, and a gate electrode 31 and a gate insulating layer 32 was formed on the substrate 30 sequentially. In the present embodiment, the preparing methods and the materials of the substrate 30, the gate electrode 31, and the gate insulating layer 32 are the same as those illustrated in Embodiment 1. In addition, in the present embodiment, the thickness of the gate electrode 31 was about 100 nm, and the thickness of the gate insulating layer 32 was about 500 nm.

Next, the evaporation process was performed on the gate insulating layer 32 to form a patterned metal layer through the same evaporation process for forming the gate electrode described in Embodiment 1, wherein the patterned metal layer was used as a source electrode 34 and a drain electrode 35, as shown in FIG. 6B. In the present embodiment, the material of the source electrode 34 and the drain electrode 35 was Au, and the thickness of the source electrode 34 and the drain electrode 35 was about 100 nm.

Finally, an organic semiconductor layer 33 was formed on the gate insulating layer 32, the source electrode 34, and the drain electrode 35 through the same process for forming the organic semiconductor layer described in Embodiment 1, as shown in FIG. 6C. In the present embodiment, the material of the organic semiconductor layer 33 is pentacene, and the thickness of the organic semiconductor layer 33 is about 100 nm.

As shown in FIG. 6C, after the aforementioned process, a bottom contact OTFT of the present embodiment was obtained, which comprises: a substrate 30; a gate electrode 31 disposed on the substrate 30; a gate insulating layer 32 disposed on the substrate 30 and covering the gate electrode 31, wherein the gate insulating layer 32 comprises silk fibroin; a source electrode 34 and a drain electrode 35 respectively located on the gate insulating layer 32; and an organic semiconductor layer 33 covering the gate insulating layer 32, the source electrode 34, and the drain electrode 35.

### Embodiment 3

### MIM Capacitor

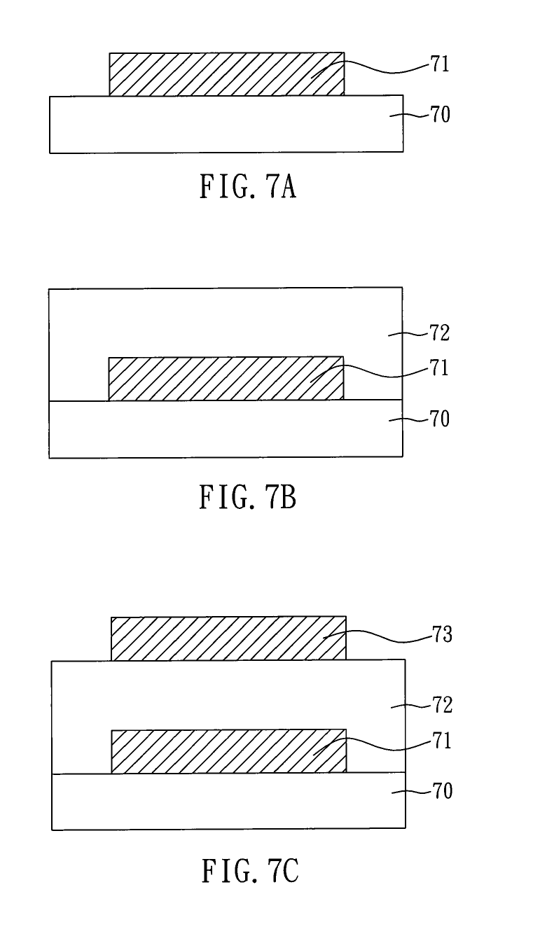

As shown in FIG. 7A, a substrate 70 was provided, and a first electrode 71 was formed on the substrate 70. In the present embodiment, the preparing method and the material of the first electrode 71 are the same as the process for forming the gate electrode illustrated in Embodiment 1. In the present embodiment, the substrate 70 was a plastic substrate, the material of the first electrode 71 was Au, and the thickness of the first electrode 71 was about 80 nm.

Next, the substrate 70 having the first electrode 71 formed thereon was dipped into the silk solution prepared in Embodiment 1 for 15 mins to coat the substrate 70 having the first electrode 71 with the silk solution. After the coating process, the substrate 70 coated with the silk solution was dried at 60° C. to form a silk film, and the silk film was used as an insulating layer 72, as shown in FIG. 7B.

Finally, the substrate 70 was placed into a vacuum chamber (not shown in the figure) with pressure of 5×10⁻⁶ torr to form a second electrode 73 with a thickness of 80 nm, as shown in FIG. 7C.

As shown in FIG. 7C, after the aforementioned process, a MIM capacitor of the present embodiment was obtained, which comprises: a substrate 70; a first electrode 71 disposed on the substrate 70; an insulating layer 72 disposed on the substrate 70 and covering the first electrode 71, wherein the insulating layer 72 comprises silk protein; and a second electrode 73 disposed on the insulating layer 72.

### [Evaluation of the Characteristics of the MIM Capacitor]

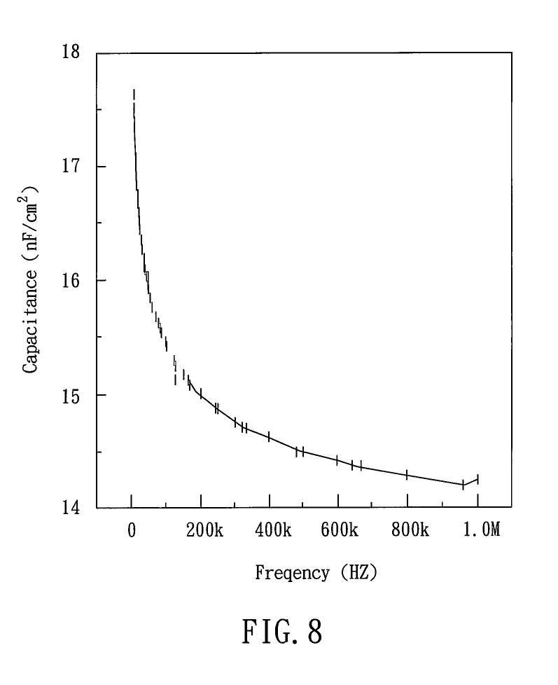

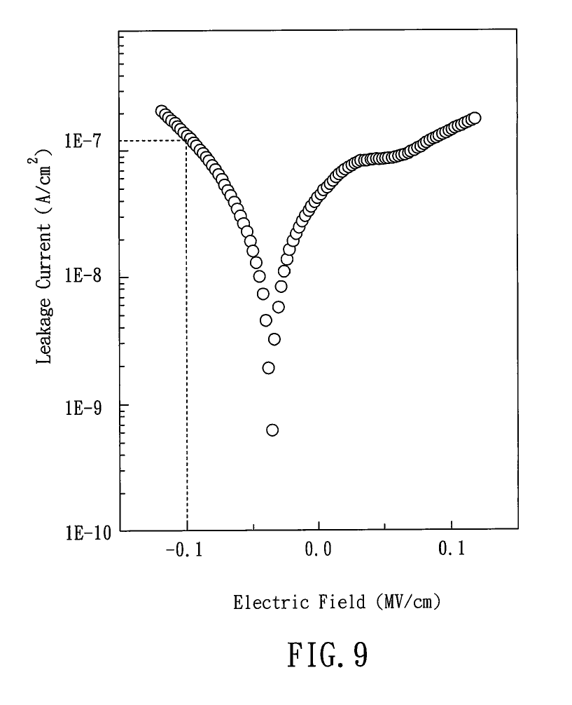

The dielectric characteristic of the MIM capacitor of the present embodiment was evaluated, and the evaluation results are shown in FIG. 8 and FIG. 9, wherein FIG. 8 is a curve showing the capacitance-frequency characteristic of the MIM capacitor, and FIG. 9 is a curve showing the electric field-leakage current characteristic of the MIM capacitor. After calculation, the dielectric constant (c) of the silk fibroin is about 7.2, and the leakage current of the MIM capacitor is about 10⁻⁷ A/cm² under −0.1 MV/cm electric field. These results indicate that the silk fibroin is a good dielectric material.

In conclusion, according to the OTFT and the MIM capacitor and the methods for manufacturing the same of the present invention, the silk fibroin is used as a dielectric material, and the gate insulating layer or the insulating layer is formed through a solution process. Hence, the complexity of the process and the production cost can be greatly decreased. Also, the process of the present invention can be used to form the OTFT and the MIM capacitor with large area. In addition, when the silk fibroin is used as the material of the gate insulating layer in the pentacene OTFT, the silk fibroin can match well with the pentacene layer. Hence, the carrier mobility of pentacene in the OTFT can be increased greatly. In addition, the electronic grade silk solution of the present invention is easily available and can be applied on various electronic devices. Furthermore, the method for manufacturing the electronic grade silk solution of the present invention is very simple, compared to the sputtering process or the vacuum deposition process. Hence, when the electronic grade silk solution of the present invention is used, not only the production cost and the complexity of the process can be decreased, but also the insulating layer with large area can be obtained.

Although the present invention has been explained in relation to its preferred embodiment, it is to be understood that many other possible modifications and variations can be made without departing from the scope of the invention as hereinafter claimed.

What is claimed is:

1. An organic thin film transistor, comprising:
a substrate;
a gate electrode disposed on the substrate;
a gate insulating layer disposed on the substrate and covering the gate electrode, wherein the gate insulating layer comprises silk protein;
an organic semiconductor layer; and
a source electrode and a drain electrode,
wherein the organic semiconductor layer, the source electrode, and the drain electrode are disposed over the gate insulating layer.

2. The organic thin film transistor as claimed in claim 1, wherein the silk protein is natural silk protein.

3. The organic thin film transistor as claimed in claim 1, wherein the silk protein is fibroin.

4. The organic thin film transistor as claimed in claim 1, wherein the gate insulating layer has a single-layered structure or a multi-layered structure.

5. The organic thin film transistor as claimed in claim 1, wherein the material of the organic semiconductor layer comprises a pentacene.

6. The organic thin film transistor as claimed in claim 1, wherein the substrate is a plastic substrate, a glass substrate, a quartz substrate, or a silicon substrate.

7. The organic thin film transistor as claimed in claim 1, wherein the organic semiconductor layer covers the entire surface of the gate insulating layer, and the source electrode and the drain electrode respectively locate on the organic semiconductor layer, when the organic thin film transistor is a top contact organic thin film transistor.

8. The organic thin film transistor as claimed in claim 1, wherein the source electrode and the drain electrode respectively locate on the gate insulating layer, and the organic semiconductor layer covers the gate insulating layer, the source electrode, and the drain electrode when the organic thin film transistor is a bottom contact organic thin film transistor.

9. A method for manufacturing an organic thin film transistor, comprising the following steps:
(A) providing a substrate;
(B) forming a gate electrode on the substrate;
(C) coating the substrate having the gate electrode formed thereon with a silk solution to obtain a gate insulating layer on the substrate and the gate electrode; and
(D) forming an organic semiconductor layer, a source electrode, and a drain electrode over the gate insulating layer.

10. The method as claimed in claim 9, wherein the step (C) comprises the following steps:
(C1) providing a silk solution;
(C2) coating the substrate having the gate electrode formed thereon with the silk solution; and
(C3) drying the silk solution coated on the substrate and the gate electrode to obtain a gate insulating layer on the substrate and the gate electrode.

11. The method as claimed in claim 9, wherein the silk solution is an aqueous solution containing natural silk protein.

12. The method as claimed in claim 9, wherein the silk solution is an aqueous solution containing fibroin.

13. The method as claimed in claim 9, wherein the material of the organic semiconductor layer comprises a pentacene.

14. The method as claimed in claim 9, wherein the substrate is a plastic substrate, a glass substrate, a quartz substrate, or a silicon substrate.

15. The method as claimed in claim 9, wherein the organic semiconductor layer covers the entire surface of the gate insulating layer, and the source electrode and the drain electrode are formed on the organic semiconductor layer to obtain a top contact organic thin film transistor, in the step (D).

16. The method as claimed in claim 9, wherein the source electrode and the drain electrode are formed on the gate insulating layer, and the organic semiconductor layer covers the source electrode, the drain electrode, and the gate insulating layer to obtain a bottom contact organic thin film transistor, in the step (D).

17. A metal-insulator-metal capacitor, comprises:
a substrate;
a first electrode disposed on the substrate;
an insulating layer disposed on the substrate and covering the first electrode, wherein the insulating layer comprises silk protein; and
a second electrode disposed on the insulating layer.

18. The metal-insulator-metal capacitor as claimed in claim 17, wherein the silk protein is natural silk protein.

19. The metal-insulator-metal capacitor as claimed in claim 17, wherein the silk protein is fibroin.

20. The metal-insulator-metal capacitor as claimed in claim 17, wherein the insulating layer has a multi-layered structure.

21. The metal-insulator-metal capacitor as claimed in claim 17, wherein the substrate is a plastic substrate, a glass substrate, a quartz substrate, or a silicon substrate.

22. A method for manufacturing a metal-insulator-metal capacitor, comprising the following steps:
(A) providing a substrate;
(B) forming a first electrode on the substrate;
(C) coating the substrate having the first electrode formed thereon with a silk solution to obtain an insulating layer on the substrate and the first electrode; and
(D) forming a second electrode on the insulating layer.

23. The method as claimed in claim 22, wherein the step (C) comprises the following steps:
(C1) providing a silk solution;
(C2) coating the substrate having the first electrode formed thereon with the silk solution; and
(C3) drying the silk solution coated on the substrate and the first electrode to obtain an insulating layer on the substrate and the first electrode.

24. The method as claimed in claim 22, wherein the silk solution is an aqueous solution containing natural silk protein.

25. The method as claimed in claim 22, wherein the silk solution is an aqueous solution containing fibroin.

26. The method as claimed in claim 22, wherein the substrate is a plastic substrate, a glass substrate, a quartz substrate, or a silicon substrate.

27. An electronic grade silk solution, comprising: silk protein, wherein the pH of the electronic grade silk solution is 3˜6.

28. The electronic grade silk solution as claimed in claim 27, wherein the electronic grade silk solution is manufactured through the following steps:
(A) placing a silkworm cocoon into an alkali solution, and heating the alkali solution to obtain silk protein;
(B) dissolving the silk protein in a phosphoric acid solution; and
(C) removing the phosphate in the phosphoric acid solution dissolving with the silk protein to obtain a silk solution.

29. The electronic grade silk solution as claimed in claim 28, wherein the step (C) is: dialyzing the phosphoric acid, which is dissolved with the silk protein, with water to remove phosphate to obtain a silk solution.

30. The electronic grade silk solution as claimed in claim 28, wherein the step (A) further comprises the following steps (A1) to (A3):
(A1) placing the silkworm cocoon into an alkali solution, and boiling the alkali solution to obtain silk protein;
(A2) taking out the silk protein from the alkali solution, and washing the silk protein; and
(A3) drying the silk protein after washing.

31. The electronic grade silk solution as claimed in claim 27, wherein the alkali solution is a solution of sodium carbonate.

32. The electronic grade silk solution as claimed in claim 27, wherein the silk protein is natural silk protein.

33. The electronic grade silk solution as claimed in claim 27, wherein the silk protein is fibroin.
